(19) United States
(12) Patent Application Publication
(10) Pub. No.: US 2014/0113337 A1
(43) Pub. Date: Apr. 24, 2014

(54) Process for Stimulating Hyaluronic Acid Production with Ascorbic Acid Derivatives and the Composition Thereof

(71) Applicant: CORUM INC., Taipei City (TW)

(72) Inventors: Wei-Yu Lin, Taipei City (TW); Ssu-Ching Wang, Taipei City (TW); Pei-Hsuan Wu, Taipei City (TW); Siao-Huei Yi, Taipei City (TW); Jun-Yi Yao, Taipei City (TW)

(73) Assignee: CORUM INC., Taipei City (TW)

(21) Appl. No.: 14/057,136

(22) Filed: Oct. 18, 2013

Related U.S. Application Data
(60) Provisional application No. 61/715,376, filed on Oct. 18, 2012.

(51) Int. Cl. C07D 307/33; C12P 19/04

(52) U.S. Cl. CPC C07D 307/33; C12P 19/04
USPC 435/101; 549/477; 252/182.12

(57) ABSTRACT

This invention discloses a process and composition for stimulating the production of hyaluronic acid. The mentioned process comprises reacting fibroblast cell with ascorbic acid derivatives. This invention provides a not only easy and un-expensive but also more efficient method for preparing safe hyaluronic acid by stimulatory in vitro.

### BACKGROUND OF THE INVENTION

1. Field of the Invention

The present invention is generally related to a process for stimulating the production of hyaluronic acid, and more particularly to a process for stimulating the production of hyaluronic acid with ascorbic acid derivatives and the composition thereof.

2. Description of the Prior Art

Hyaluronic Acid (HA), also called Hyaluronan or sodium hyaluronate, is a naturally occurring, highly hydrated linear polymer composed of the repeating disaccharide D-glucuronic acid-beta-1,3-N-acetylglucosamine-beta-1,4. Molecular weight of HA is between 1000 kDa˜10000 kDa. It is found in large concentrations in the extracellular matrix (ECM), which is the fluid-filled space between cells. HA locks moisture into the ECM, keeping collagen and elastin moist and promoting a youthful appearance.

HA is widely used in therapeutics and cosmetology. The most popular source to obtain HA is as the following: animal organ extraction, microorganism fermentation, and chemical synthesis. All the mentioned sources of HA are extrinsic to human body, so that the mentioned HA can be possibly dangerous to human body and cannot stay long time in human body.

In view of the above matter, developing a novel process and a novel composition for stimulating the production of hyaluronic acid having the advantage of high stimulating yield of HA, and high safety to human body is an important task for the industry.

### SUMMARY OF THE INVENTION

In light of the above background, in order to fulfill the requirements of the industry, the present invention provides a novel process and a novel composition for stimulating the production of hyaluronic acid having the advantage of high stimulating yield of hyaluronic acid, and high safety to human body, so that the production of hyaluronic acid can be approached.

One object of the present invention is to provide a process and a novel composition for stimulating the production of hyaluronic acid to be more safety by producing hyaluronic acid inside human body.

Another object of the present invention is to provide a process and a novel composition for stimulating the production of hyaluronic acid to improve the cosmetic/therapeutical result with hyaluronic acid by stimulating the production of hyaluronic acid at the fibroblast cell at the target area of the human body.

Accordingly, the present invention discloses a process for stimulating the production of hyaluronic acid. The mentioned process for stimulating the production of hyaluronic acid comprises a step of reacting fibroblast cell with ascorbic acid derivatives. According to this invention, through stimulating by ascorbic acid derivatives, the production of hyaluronic acid can be approached more efficiently and more safety. Preferably, according to this invention, the production of hyaluronic acid can be stimulated efficiently by small amount of ascorbic acid derivatives.

In one preferred embodiment of this invention, the process for stimulating the production of hyaluronic acid can employ the ascorbic acid derivatives with the following formula to stimulate fibroblast cell to prepare hyaluronic acid.

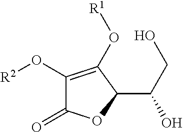

In one preferred embodiment of this invention, the process for stimulating the production of hyaluronic acid can employ the ascorbic acid derivatives with the concentration of 7500 ppm-1.25 ppm to stimulate fibroblast cell to prepare hyaluronic acid.

In one preferred embodiment of this invention, the process for stimulating the production of hyaluronic acid can employ 3-O-alkyl ascorbic acid to stimulate fibroblast cell to prepare hyaluronic acid.

In one preferred embodiment of this invention, the process for stimulating the production of hyaluronic acid can employ 3-O-ethyl ascorbic acid to stimulate fibroblast cell to prepare hyaluronic acid.

This present invention also discloses a composition for stimulating the production of hyaluronic acid. The mentioned composition comprises ascorbic acid derivatives. The ascorbic acid derivatives is employed for stimulating fibroblast cell to prepare hyaluronic acid. The general formula of the ascorbic acid derivatives is as the following:

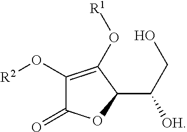

In one preferred embodiment of this invention, the concentration of the ascorbic acid derivatives in the composition for stimulating the production of hyaluronic acid can be 75000 ppm-1.25 ppm.

In one preferred embodiment of this invention, the ascorbic acid derivatives in the composition for stimulating the production of hyaluronic acid can be 3-O-alkyl ascorbic acid.

### BRIEF DESCRIPTION OF THE DRAWINGS

The present disclosure can be described by the embodiments given below. It is understood, however, that the embodiments below are not necessarily limitations to the present disclosure, but are used to a typical implementation of the invention.

FIG. 1 presents the analysis diagram of the produced concentration of hyaluronic acid with mouse fibroblast (3T3) stimulated by ascorbic acid derivatives with the process according to this specification and measured in Echelon HA-ELISA;

FIG. 2 presents the analysis diagram of the produced concentration of hyaluronic acid with human fibroblast (HS68) stimulated by ascorbic acid derivatives with the process according to this specification and measured in Echelon HA-ELISA;

FIG. 3 presents the analysis diagram of the produced concentration of hyaluronic acid with mouse fibroblast (3T3) stimulated by ascorbic acid derivatives with the process according to this specification and measured in R&D HA-ELISA;

FIG. 4 presents the analysis diagram of the produced concentration of hyaluronic acid with human fibroblast (HS68) stimulated by ascorbic acid derivatives with the process according to this specification and measured in R&D HA-ELISA; and

FIG. 5 presents the analysis diagram of the produced concentration of hyaluronic acid with mouse fibroblast (3T3) stimulated by ascorbic acid (AA) with the process according to this specification and measured in R&D HA-ELISA.

### DESCRIPTION OF THE PREFERRED EMBODIMENTS

What probed into the invention is a process for stimulating the production of hyaluronic acid and the composition thereof. Detailed descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater details in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

One preferred embodiment according to this specification discloses a process for stimulating the production of hyaluronic acid. According to this embodiment, the mentioned process comprises reacting fibroblast cell with ascorbic acid derivatives. The general formula of the mentioned ascorbic acid derivatives is as the following.

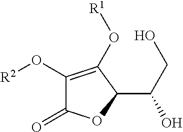

In the above-mentioned general formula, R¹ and R² are independently selected from the group consisting of the following: H atom, C1-C2 alkyl group, C3-C20 cycloalkyl group, C1-C20 heterocycloalkyl group, C1-C20 alkoxy group, C2-C20 acyl group, C6-C20 aryl group, C1-C20 heterocyclic aromatic group, C3-C20 cycloalkenyl group. In the above-mentioned general formula, R¹ and R² will not be H atom at the same time. In one preferred example of this embodiment, the mentioned ascorbic acid derivatives is 3-O-alkyl-ascorbic acid. According to this example, preferably, the alkyl group of the 3-O-alkyl-ascorbic acid is C2 to C10 alkyl group. In one preferred example of this embodiment, the concentration of the mentioned ascorbic acid derivatives is about 7500 ppm-1.25 ppm. In another preferred example of this embodiment, the concentration of the mentioned ascorbic acid derivatives is about 2500 ppm-1.25 ppm.

Another preferred embodiment according to this specification discloses a composition for stimulating the production of hyaluronic acid. According to this embodiment, the mentioned composition comprises ascorbic acid derivatives. The general formula of the mentioned ascorbic acid derivatives is as the following.

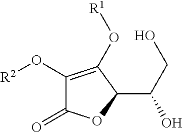

In the above-mentioned general formula, R¹ and R² are independently selected from the group consisting of the following: H atom, C1-C20 alkyl group, C3-C20 cycloalkyl group, C1-C20 heterocycloalkyl group, C1-C20 alkoxy group, C2-C20 acyl group, C6-C20 aryl group, C1-C20 heterocyclic aromatic group, C3-C20 cycloalkenyl group. In the above-mentioned general formula, R¹ and R² will not be H atom at the same time. In one preferred example of this embodiment, the mentioned ascorbic acid derivative is 3-O-alkyl-ascorbic acid. According to this example, preferably, the alkyl group of the 3-O-alkyl-ascorbic acid is C2 to C10 alkyl group.

According to this embodiment, the ascorbic acid derivatives is employed for reacting with fibroblast cell, and stimulating the fibroblast cell producing hyaluronic acid. In one preferred example of this embodiment, the concentration of the ascorbic acid derivatives is about 7500 ppm-1.25 ppm. In another preferred example of this embodiment, the concentration of the ascorbic acid derivatives is about 2500 ppm-1.25 ppm.

In order to illustrate this specification, the preferred examples of the process for stimulating the production of hyaluronic acid and the composition for stimulating the production of hyaluronic acid according to the invention are described in the following. In the following examples, the process for stimulating the production of hyaluronic acid is applied in mouse cell and human cell step by step. However, the scope of the invention should be based on the claims, but is not restricted by the following examples.

### Example 1

### General Procedure of Stimulating the Production of Hyaluronic Acid with Mouse Fibroblast

(hereinafter 3-O-ethyl-ascorbic acid is used for illustration, and this specification is not limited by the following examples)

Materials:

Cell Line: 3T3 (BCRC 60071, purchased from Bioresource Collection and Research Center)

Rinse Buffer: Sterile D-PBS (Dulbecco's phosphate buffered saline, purchased from Corning Incoperated and Caisson Labs)

Complete Medium (as positive control): DMEM medium with 10% Fetal Bovine Serum (DMEM purchased from Corning Incoperated and Caisson Labs; FBS purchased from Caisson Labs)

Starvation Medium (as negative control): DMEM medium with 0.1% Bovine Serum (DMEM purchased from Corning Incoperated and Caisson Labs; FBS purchased from Caisson Labs)

Testing material: 3-O-ethyl-ascorbic acid

Hyaluronan Enzyme-Linked Immunosorbent Assay Kit (HA-ELISA) (Brand: Echelon/Cat No: K-1200)

Cell Seeding:

Complete medium is used in cell seeding stage. The confluent 3T3 cells are rinsed with sterile D-PBS, and trypsinize all cells and perform cell counting. Then, dilute the cell density to 4E+04/mL and seed 0.5 mL cell liquid (equals to 2E+04 cells) each well in 24 well plates. Incubate 3T3 cells at 37° C., 5% CO₂ for about 24 hours.

Controls/Testing Material Treatment:

Starvation medium is used in this stage. The 24-hour-cultured 3T3 cells are rinsed with sterile D-PBS and discarded rinse buffer. Controls and testing materials in different concentrations are respectively added into the 3T3 cells in 24 well plates. Then, the 3T3 cells are incubated at 37° C., with 5% CO₂ for about 72 hours.

Quantitation of Hyaluronic Acid:

After incubating for about 72 hours, transfer some supernatant of cells treated with controls and testing materials into new containers. Centrifuge the supernatants at 2000 rpm for 5˜10 minutes. The hyaluronic acid content of the centrifuged supernatants are respectively determined by Hyaluronan Enzyme-Linked Immunosorbent Assay Kit, and calculated with the following Formula 1.

$$Percentage to Control ( \% ) = \frac{\begin{matrix}HA Content of Positive \\ Control or testing materials\end{matrix}}{HA Content of Negative Control} \times 100 \% \qquad Formula 1$$

The following Table 1 lists several repeat test results according to the above mentioned experiments. As shown in Table 1, comparing to the negative control, the 2500 ppm 3-O-ethyl-ascorbic acid can maximally stimulate hyaluronic acid production to 230.6%. It can also be found from Table 1 that when the concentration of 3-O-ethyl-ascorbic acid is 10-5000 ppm, the stimulated hyaluronic acid production can be efficiently increased by the ascorbic acid derivatives. That is, according to this specification, the stimulatory of producing hyaluronic acid can be approached efficiently with few ascorbic acid derivatives.

Due to the cell viability were inhibited by 3-O-ethyl-ascorbic acid with the concentration larger than 7500 ppm, this group didn't show stimulating ability for hyaluronic acid production. And, ascorbic acid derivatives of those concentrations might become cytotoxicity to the cell so that the secretion of hyaluronic acid will be decreased.

TABLE 1
Data Ayalysis of Hyaluronic Acid Content
Stimulated by 3-O-ethyl-ascorbic acid in mouse fibroblast (3T3)

| Sample name/ | | | | | | | |
|---|---|---|---|---|---|---|---|
| concentration | | Entry-1 | Entry-2 | Entry-3 | Entry-4 | Average | Stdev |
| Positive Control | | 796.8% | >max* | 721.8% | 524.0% | 680.8% | 140.9% |
| Negative Control | | 100.0% | 100.0% | 100.0% | 100.0% | 100.0% | 0.0% |
| 3-O-ethyl- | 10000 | 123.0% | 122.4% | 61.1% | 51.0% | 89.4% | 38.7% |
| ascorbic acid (ppm) | 7500 | 64.9% | 123.2% | 120.1% | 55.9% | 91.0% | 35.6% |
| | 5000 | 110.3% | 214.1% | 161.9% | 109.6% | 149.0% | 49.9% |
| | 2500 | 161.6% | 193.4% | 74.2% | 230.6% | 165.0% | 66.7% |
| | 1000 | 216.8% | 212.8% | 80.8% | 85.2% | 148.9% | 76.1% |
| | 100 | 204.5% | 150.2% | 86.0% | 91.5% | 133.1% | 55.8% |
| | 10 | 203.7% | 197.3% | 92.9% | 164.6% | 164.6% | 50.8% |

*The concentration of sample is out of standard curve.

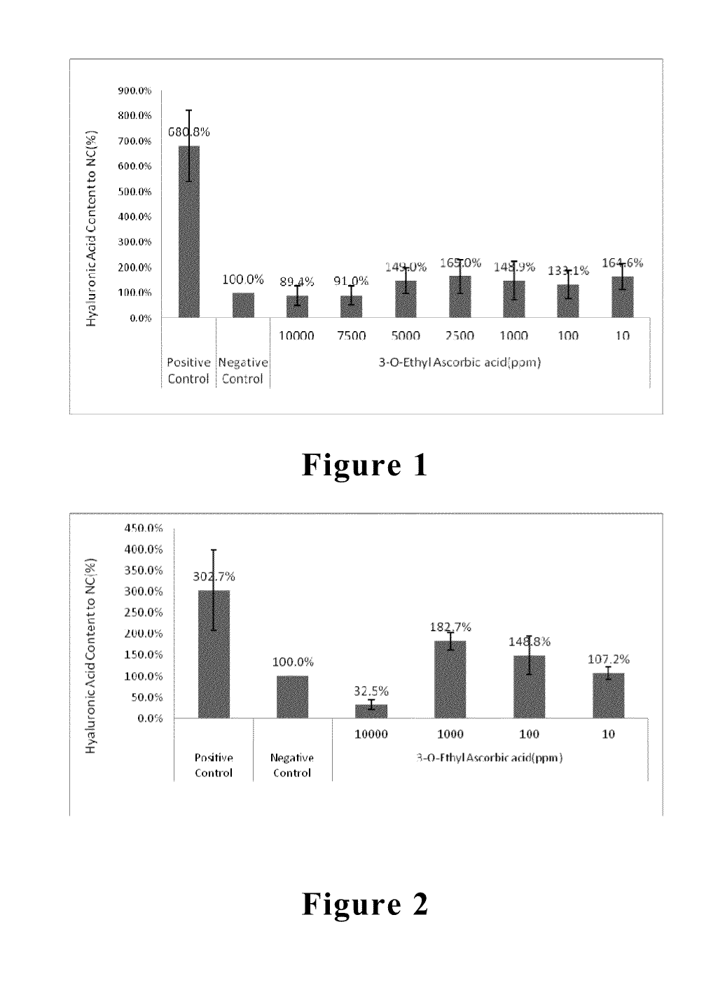

FIG. 1 shows the stimulatory effect on Hyaluronic Acid secretion by mouse fibroblast (3T3) of the Table 1. From FIG. 1, it is obviously that the ascorbic acid derivatives can improve the stimulating hyaluronic acid production.

### Example 2

### General Procedure of Stimulating the Production of Hyaluronic Acid with Human Fibroblast

(hereinafter 3-O-ethyl-ascorbic acid is used for illustration, and this specification is not limited by the following examples)

Materials:

Cell Line: Hs68 (BCRC 60038, purchased from Bioresource Collection and Research Center)

Rinse Buffer: Sterile D-PBS (Dulbecco's phosphate buffered saline, purchased from Corning Incoperated and Caisson Labs)

Complete Medium (as positive control): DMEM medium with 10% Fetal Bovine Serum (DMEM purchased from Corning Incoperated and Caisson Labs; FBS purchased from Caisson Labs)

Starvation Medium (as negative control): DMEM medium with 0.1% Fetal Bovine Serum (DMEM purchased from Corning Incoperated and Caisson Labs; FBS purchased from Caisson Labs)

Testing material: 3-O-ethyl-ascorbic acid

Hyaluronan Enzyme-Linked Immunosorbent Assay Kit (HA-ELISA) (Brand: Echelon/Cat No: K-1200)

Cell Seeding:

Complete medium is used in cell seeding stage. The confluent Hs68 cells are rinsed with sterile D-PBS, and trypsinize all cells and perform cell counting. Then, dilute the cell density to 4E+04/mL and seed 0.5 mL cell liquid (equals to 2E+04 cells) each well in 24 well plates. Incubate Hs68 cells at 37° C., 5% CO₂ for about 24 hours.

Controls/Testing Material Treatment:

Starvation medium is used in this stage. The 24-hour-cultured Hs68 cells are rinsed with sterile D-PBS and discarded rinse buffer. Controls and testing materials in different concentrations are respectively added into the Hs68 cells in 24 well plates. Then, the Hs68 cells are incubated at 37° C., with 5% CO₂ for about 72 hours.

Quantitation of Hyaluronic Acid:

After incubating for about 72 hours, transfer some supernatant of cells treating with controls and testing materials into new containers. Centrifuge the supernatants at 2000 rpm for 5˜10 minutes. The hyaluronic acid content of the centrifuged supernatants are respectively determined by Hyaluronan Enzyme-Linked Immunosorbent Assay Kit, and calculated with the following Formula 2.

$$Percentage to Control ( \% ) = \frac{\begin{matrix}HA Content of Positive \\ Control or testing materials\end{matrix}}{HA Content of Negative Control} \times 100 \% \qquad Formula 2$$

The following Table 2 lists several repeat test results according to the above mentioned experiments. As shown in Table 2, comparing to the negative control, the 1000 ppm 3-O-ethyl-ascorbic acid can maximally stimulate hyaluronic acid production to 205.4%. When the concentration of 3-O-ethyl-ascorbic acid is 1000-10 ppm, it can be found from the mentioned Table 2 that the stimulated hyaluronic acid production is dose-dependently upon the concentration of the ascorbic acid derivatives. Additionally, as shown in Table 2, according to this specification, the stimulatory of producing hyaluronic acid can be approached efficiently with few ascorbic acid derivatives.

Due to the cell viability were inhibited by 10000 ppm 3-O-ethyl-ascorbic acid, this group didn't show dose dependent trend in stimulating hyaluronic acid production. Moreover, the ascorbic acid derivatives might be cytotoxicity for producing hyaluronic acid.

TABLE 2
Data Ayalysis of Hyaluronic Acid Content
Stimulated by 3-O-ethyl-ascorbic acid in human fibroblast (Hs68)

| Sample Name | | Nega- | | | | |
|---|---|---|---|---|---|---|
| Sample | Positive | tive | 3-O-ethyl-ascorbic acid (ppm) | | | |
| Concentration | Control | Control | 10000 | 1000 | 100 | 10 |
| Entry-1 | 282.2% | 100.0% | 19.4% | 170.1% | 130.3% | 123.3% |
| Entry-2 | 407.3% | 100.0% | 40.6% | 172.6% | 115.1% | 105.7% |
| Entry-3 | 218.7% | 100.0% | 37.4% | 205.4% | 200.9% | 92.7% |
| Average | 302.7% | 100.0% | 32.5% | 182.7% | 148.8% | 107.2% |
| Stdev | 96.0% | 0.0% | 11.4% | 19.7% | 45.8% | 15.3% |

FIG. 2 shows the stimulatory effect on Hyaluronic Acid secretion by human fibroblast of the Table 2. From FIG. 2, it can be found that the dose-dependent trend between the ascorbic acid concentration and the producing hyaluronic acid is obviously.

### Example 3

### Test of Stimulating the Production of Hyaluronic Acid with Mouse Fibroblast

In this example, we try another brand HA-ELISA kit (R&D; Cat No. DHYAL0) to figure out the reproducibility of the stimulatory effect. Excluding replacing the HA-ELISA kit, the materials and the procedures can be referred to the above Example 1.

The following Table 3 lists several repeat test results according to the above mentioned experiments. As shown in Table 3, comparing to the negative control, the 5000 ppm 3-O-ethyl-ascorbic acid can maximally stimulate hyaluronic acid production to 240.5%. When the concentration of 3-O-ethyl-ascorbic acid is 5000-5 ppm, it can be found from the Table 3 that the stimulated hyaluronic acid production is dose-dependently upon the concentration of the ascorbic acid derivatives. Additionally, referred to Table 3, according to this specification, the stimulatory of producing hyaluronic acid can be approached efficiently with few ascorbic acid derivatives.

TABLE 3
Data Ayalysis of Hyaluronic Acid Content
Stimulated by 3-O-ethyl-ascorbic acid in mouse fibroblast (3T3)

| Sample name/ | | | | | | |
|---|---|---|---|---|---|---|
| concentration | | Entry-1 | Entry-2 | Entry-3 | Average | Stdev |
| Positive Control | | 963.2% | 478.5% | 497.6% | 646.4% | 274.5% |
| Negative Control | | 100.0% | 100.0% | 100.0% | 100.0% | 0.0% |
| 3-O-ethyl- | 10000 | 64.7% | 117.1% | 145.3% | 109.0% | 40.9% |
| ascorbic | 7500 | 119.9% | 130.8% | 180.7% | 143.8% | 32.4% |
| acid (ppm) | 5000 | 131.8% | 180.4% | 240.5% | 184.2% | 54.5% |
| | 2500 | 148.7% | 176.3% | 152.8% | 159.3% | 14.9% |
| | 1000 | 111.0% | 188.3% | 234.1% | 177.8% | 62.2% |
| | 100 | 166.8% | 155.7% | 191.2% | 171.2% | 18.2% |
| | 10 | 101.0% | 158.1% | 182.2% | 147.1% | 41.7% |
| | 7.5 | 115.9% | 143.8% | 148.1% | 135.9% | 17.5% |
| | 5 | 78.2% | 152.4% | 180.6% | 137.1% | 52.9% |
| | 2.5 | 119.7% | 135.2% | 182.8% | 145.9% | 32.9% |
| | 1.25 | 100.0% | 156.8% | 172.0% | 143.0% | 37.9% |

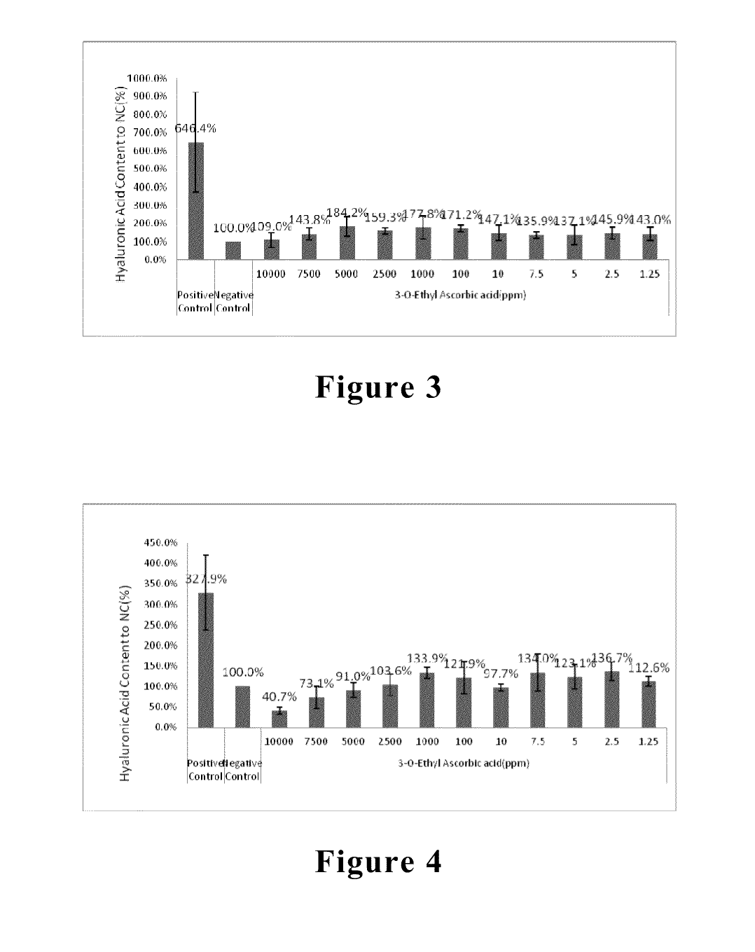

FIG. 3 shows the stimulatory effect on Hyaluronic Acid secretion by mouse fibroblast (3T3) of the Table 3. From FIG. 3, it is obviously that the ascorbic acid derivatives can improve the stimulating hyaluronic acid production, and Hyaluronic Acid stimulating effect of the ascorbic acid derivatives is obviously, even using kits from different brands.

### Example 4

### Test of Stimulating the Production of Hyaluronic Acid with Human Fibroblast

In this example, we try another brand HA-ELISA kit (R&D; Cat No. DHYAL0) to study the reproducibility of the stimulatory effect. Excluding replacing the HA-ELISA kit, the materials and the procedures can be referred to the above Example 2.

The following Table 4 lists several repeat test results according to the above mentioned experiments. As shown in Table 4, comparing to the negative control, the 100 ppm 3-O-ethyl-ascorbic acid can maximally stimulate hyaluronic acid production to 174.9%. According to Table 4, when the concentration of 3-O-ethyl-ascorbic acid is controlled at 1000 ppm, 100 ppm, and 7.5 ppm, the corresponding stimulated hyaluronic acid production is 133.9%, 121.9%, and 134.0%. Preferably, when the concentration of 3-O-ethyl-ascorbic acid is 2.5 ppm, the stimulated hyaluronic acid production still can achieve 136.7%. That is, referred to Table 4, the stimulatory of producing hyaluronic acid can be approached efficiently with few ascorbic acid derivatives. It can be found from the Table 4 that when the concentration of 3-O-ethyl-ascorbic acid is about 1000-10 ppm, the stimulated hyaluronic acid production is dose-dependently upon the concentration of the ascorbic acid derivatives.

TABLE 4
Data Ayalysis of Hyaluronic Acid Content
Stimulated by 3-O-ethyl-ascorbic acid in human fibroblast (HS68)

| Sample name/ | | | | | | | |
|---|---|---|---|---|---|---|---|
| concentration | | Entry-1 | Entry-2 | Entry-3 | Entry-4 | Average | Stdev |
| Positive Control | | 410.8% | 359.5% | 342.9% | 198.2% | 327.9% | 91.1% |
| Negative Control | | 100.0% | 100.0% | 100.0% | 100.0% | 100.0% | 0.0% |
| 3-O-ethyl-ascorbic | 10000 | 50.9% | 32.1% | 37.2% | 42.4% | 40.7% | 8.0% |
| acid (ppm) | 7500 | 114.2% | 54.6% | 67.1% | 56.4% | 73.1% | 28.0% |
| | 5000 | 110.0% | 70.6% | 103.2% | 80.3% | 91.0% | 18.6% |
| | 2500 | 99.2% | 96.7% | 78.7% | 140.0% | 103.6% | 25.9% |
| | 1000 | 125.4% | 134.8% | 122.6% | 152.7% | 133.9% | 13.6% |
| | 100 | 123.5% | 104.5% | 84.8% | 174.9% | 121.9% | 38.7% |
| | 10 | 89.1% | 101.2% | 91.2% | 109.5% | 97.7% | 9.4% |
| | 7.5 | 67.9% | 162.0% | 157.3% | 148.8% | 134.0% | 44.4% |
| | 5 | 110.6% | 110.9% | 103.8% | 167.0% | 123.1% | 29.5% |
| | 2.5 | 149.7% | 125.3% | 111.4% | 160.5% | 136.7% | 22.4% |
| | 1.25 | 102.1% | 126.8% | 102.4% | 119.0% | 112.6% | 12.3% |

FIG. 4 shows the stimulatory effect on Hyaluronic Acid secretion by human fibroblast of the Table 4. From FIG. 4, it can be found that the dose-dependent trend between the ascorbic acid concentration and the producing hyaluronic acid is obviously even using kits from different Brands.

### Example 5

### Test of Stimulating the Production of Hyaluronic Acid with Ascorbic Acid (AA) in Mouse Fibroblast

In this example, we try to figure out whether the stimulatory effect also can be found on ascorbic acid. Excluding replacing the HA-ELISA kit to another brand HA-ELISA kit (R&D; Cat No. DHYAL0) and replacing the testing material to ascorbic acid, the other materials and the procedures can be referred to the above Example 1.

The following Table 5 lists several repeat test results according to the above mentioned experiments. As shown in Table 5, comparing to the negative control, the 2-100 ppm ascorbic acid can NOT stimulate hyaluronic acid production. Furthermore, in some entries, the mentioned ascorbic acid will decrease the concentration of hyaluronic acid. That is, comparing with the disclosure of this specification, ascorbic acid cannot be employed for the stimulatory of producing hyaluronic acid.

TABLE 5
Data Ayalysis of Hyaluronic Acid Content
Stimulated by ascorbic acid in mouse fibroblast (3T3)

| Sample name/ | | | | | | |
|---|---|---|---|---|---|---|
| concentration | | Entry-1 | Entry-2 | Entry-3 | Average | Stdev |
| Positive Control | | 460% | 550% | 647% | 552% | 93% |
| Negative Control | | 100% | 100% | 100% | 100% | 0% |
| Ascorbic | 100 | 87% | 93% | 94% | 91% | 4% |
| Acid | 50 | 105% | 100% | 102% | 102% | 2% |
| (ppm) | 10 | 93% | 108% | 101% | 101% | 7% |
| | 2 | 99% | 64% | 119% | 94% | 27% |

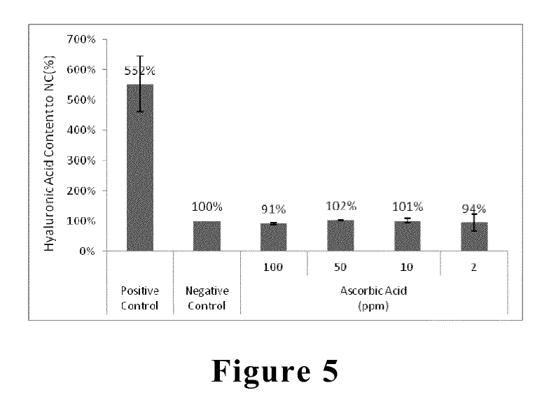

FIG. 5 shows the stimulatory effect on Hyaluronic Acid secretion by human fibroblast of the Table 5. From FIG. 5, it can be found that ascorbic acid cannot be helpful to the stimulatory of producing hyaluronic acid.

In summary, this application has reported a process and composition for stimulating the production of hyaluronic acid. According to this application, the mentioned process comprises a step of reacting fibroblast cells with ascorbic acid derivatives. The mentioned composition comprises ascorbic acid derivatives for stimulating the production of hyaluronic acid. Through treating mouse or human fibroblast cells with the ascorbic acid derivatives, the production of hyaluronic acid can be efficiently increased. The mentioned process and composition for stimulating the production of hyaluronic acid of this application can stimulate the production of hyaluronic acid of mouse and human fibroblast cell in vitro. More preferably, the stimulating production of hyaluronic acid of this application can be approached with few amount ascorbic acid derivatives. That is, this application provides a more safe and efficient method for increasing hyaluronic acid in cells for therapeutics and cosmetology.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A process for stimulating the production of hyaluronic acid, comprising:
a step of reacting fibroblast cell with ascorbic acid derivatives, wherein the general formula of said ascorbic acid derivatives is as the following: 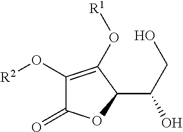
wherein R¹ and R² are independently selected from the group consisting of the following: H atom, C1-C20 alkyl group, C3-C20 cycloalkyl group, C1-C20 heterocycloalkyl group, C1-C20 alkoxy group, C2-C20 acyl group, C6-C20 aryl group, C1-C20 heterocyclic aromatic group, C3-C20 cycloalkenyl group, wherein R¹ and R² are not both H atom at the same time.

2. The process for stimulating the production of hyaluronic acid according to claim 1, wherein the concentration range of said ascorbic acid derivatives is 7500 ppm-1.25 ppm.

3. The process for stimulating the production of hyaluronic acid according to claim 1, wherein the concentration range of said ascorbic acid derivatives is 2500 ppm-1.25 ppm.

4. The process for stimulating the production of hyaluronic acid according to claim 1, wherein said ascorbic acid derivatives is 3-O-alkyl-ascorbic acid.

5. The process for stimulating the production of hyaluronic acid according to claim 3, wherein the alkyl group of the 3-O-alkyl-ascorbic acid is C2 to C10 alkyl group.

6. The process for stimulating the production of hyaluronic acid according to claim 1, wherein said ascorbic acid derivatives is 3-O-ethyl-ascorbic acid.

7. A composition for stimulating the production of hyaluronic acid, comprising:
ascorbic acid derivatives, wherein said ascorbic acid derivatives is employed for reacting with fibroblast cell and stimulating the fibroblast cell to produce hyaluronic acid, wherein the general formula of said ascorbic acid derivatives is as the following: 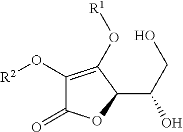
wherein R¹ and R² are independently selected from the group consisting of the following: H atom, C1-C20 alkyl group, C3-C20 cycloalkyl group, C1-C20 heterocycloalkyl group, C1-C20 alkoxy group, C2-C20 acyl group, C6-C20 aryl group, C1-C20 heterocyclic aromatic group, C3-C20 cycloalkenyl group, wherein R¹ and R² are not both H atom at the same time.

8. The composition for stimulating the production of hyaluronic acid according to claim 7, wherein the concentration range of said ascorbic acid derivatives is 7500 ppm-1.25 ppm.

9. The composition for stimulating the production of hyaluronic acid according to claim 7, wherein the concentration range of said ascorbic acid derivatives is 2500 ppm-1.25 ppm.

10. The composition for stimulating the production of hyaluronic acid according to claim 7, wherein said ascorbic acid derivatives is 3-O-alkyl-ascorbic acid.

11. The process for stimulating the production of hyaluronic acid according to claim 10, wherein the alkyl group of the 3-O-alkyl-ascorbic acid is C2 to C10 alkyl group.

12. The composition for stimulating the production of hyaluronic acid according to claim 7, wherein said ascorbic acid derivatives is 3-O-ethyl-ascorbic acid.
